(19) United States
(12) Patent Application Publication
(10) Pub. No.: US 2010/0076465 A1
(43) Pub. Date: Mar. 25, 2010

(54) FIBRIN-BASED NERVE REPAIR CONDUIT AND METHOD OF PRODUCING THE SAME

(75) Inventors: Mikael Wiberg, Umea (SE); Giorgio Terenghi, Manchester (GB)

Correspondence Address: BACON & THOMAS, PLLC, 625 SLATERS LANE, FOURTH FLOOR, ALEXANDRIA, VA, 22314-1176, US

(73) Assignee: AXONGEN AB, UMEA (SE)

(21) Appl. No.: 12/451,506

(22) PCT Filed: May 15, 2008
(86) PCT No.: PCT/SE2008/050574

§ 371 Date: Nov. 16, 2009

Related U.S. Application Data
(60) Provisional application No. 60/924,434, filed on May 15, 2007.

(30) Foreign Application Priority Data
May 15, 2007 (SE) 0701168-7

(51) Int. Cl. A61B 17/00; A61K 9/00; A61K 38/48; A61K 35/12; A61P 25/00; B29C 41/42

(52) U.S. Cl. 606/152; 424/423; 424/94.64; 424/93.7

(57) ABSTRACT

A bioresorbable fibrin-based nerve repair conduit produced from tissue glue is disclosed. The nerve repair conduit may be in the form of a sheet or a tube, and may additionally comprise a serine protease, and/or Factor XIII and/or calcium ions. The serine protease is chosen from the group consisting of trombin, plasmin, elastases, and plasminogen activators, or combinations thereof. The nerve repair conduit may moreover be loaded with Schwann cells and/or Stem cells and/or growth factors, for better nerve regeneration. Further, a method of producing the above-mentioned nerve repair conduit is provided, comprising curing fibrinogen and serine protease containing fluids in the form of a fibrinogen-containing tissue glue, in a mould equipped with a central shaft creating a channel in the nerve repair conduit when removed. Moreover, use of a fibrinogen-based tissue glue for the preparation of a fibrin-based nerve repair conduit is described. A method of treating nerve damage by placing a nerve repair conduit according to the invention around at least one nerve stump and allowing the nerve repair conduit to guide the nerve stump during regeneration is also provided.

The current invention relates to a fibrin-based nerve repair conduit produced from tissue glue and a method of producing the same. The invention is particularly concerned with a nerve repair conduit for peripheral nerve repair. The invention further relates to the use of glue-forming fibrinogen and serine protease containing fluids, for the preparation of a nerve repair conduit as well as treatment of nerve damage.

### BACKGROUND

Nerve injuries without defect or with a short gap are usually treated by end-to-end coaptation. The normal nerve segments proximal and distal to the site of neurorrhaphy are sufficiently extensible to compensate for the short defects. If there is a longer defect, a neurorrhaphy without tension at the site of the repair cannot be performed¹ and surgical repair of nerve gaps greater than 20 mm is commonly achieved by autologous nerve grafts. An autologous nerve graft provides Schwann cells (SC), growth factors and basal lamina components and is the current gold standard but has associated problems. Scarring, neuroma formation and poor sensory function recovery are common consequences. Previous studies have reported a poor recovery of sensation as well as only partially recovered motor function in most cases²⁻⁵. Autologous alternatives have been sought and include autologous conduits such as venous or arterial conduit grafts but these did not show any functional benefits compared with standard nerve grafts^{6, 7}. Peripheral nerve allografts using cadaver tissue have been tested, but they have many limitations especially because of the undesirable long-term immunosuppressive therapy required⁸. In order to achieve a better clinical outcome, several synthetic nerve repair conduits have been studied to replace nerve autografts and allografts. Non-degradable materials such as silicone^{9, 10}, polytetrafluorethylene (PTFE) and polypyrrole (PPY) have been thought to provide a permissive environment for outgrowing axons allowing the supportive supply of neurotropic factors and SC^{11, 12}. However, it was noted that compression syndromes often occurred because of their non-degradable nature and their inability to adapt to the nerve growth and maturation^{5, 13, 14}. Moreover, increased scarring and irritation of the patient has been described¹⁵. Increasingly, synthetic nerve repair conduits used for bridging neural gaps are made of biodegradable or bioresorbable materials^{16, 17}. Among these, poly 3-hydroxybutyrate (PHB) nerve repair conduits have gained particular interest and have been extensively investigated. PHB nerve repair conduits have a soft malleable consistency, good tensile strength and flexibility. PHB nerve repair conduits show early vascularisation after implantation and are resorbed over a period of two years¹⁸. These above-mentioned nerve repair conduits have a rather long resorption time, whereas an optimal nerve repair conduit should dissolve within weeks to a few months, having supported the regenerating axons to cross the nerve gap and allowing neurotrophic factors to penetrate during the early phase of regeneration. It is desirable to obtain nerve repair conduits for nerve repair with improved resorbability.

### DESCRIPTION OF THE INVENTION

The current invention pertains to a novel nerve repair conduit based on fibrin. Such a nerve repair conduit is in accordance with the invention prepared from a fibrinogen-containing fluid, i.e. a. fibrin glue as defined herein. Using a rodent sciatic nerve injury model (10 mm gap) the extent of nerve regeneration through fibrin and PHB nerve repair conduits was compared. After two weeks, nerve repair conduits containing proximal and distal stumps were harvested. Both types of nerve repair conduit presented full tissue integration and were completely intact. Immunohistochemistry using the axonal marker PGP9.5 showed a superior nerve regeneration distance in the fibrin-based nerve repair conduit compared with PHB (4.mm vs 1.9 mm). Schwann cell intrusion (S100 staining) was similarly enhanced in the fibrin-based nerve repair conduits, both from the proximal (4.2 mm vs 2.1 mm) and distal ends (3.2 mm vs 1.7 mm). These findings indicate a significant advantage of the new fibrin-based nerve repair conduit for the initial phase of peripheral nerve regeneration.

According to a first aspect of the current invention, a bioresorbable fibrin-based nerve repair conduit produced from tissue glue is provided. The term “bioresorbable” is used herein as an expression for a material that is resorbed by the body within a time period of a few months, when placed therein. The term “fibrin-based” is used herein to indicate that the main component of the conduit is fibrin but other components, especially blood or plasma components, may be present in the conduit. Tissue glue is used herein as a reference to a fibrinogen-containing mixture that is converted into fibrin. The tissue glue may be of endogenous origin, whereby it would be manufactured from plasma components of the patient's own blood, with the facultative addition of additional components, dependent on the cleavage system used for the conversion of fibrinogen into fibrin. Endogenous plasma components have the advantage of minimizing the risk for transfer of contaminants such as infectious agents. Moreover, endogenous products reduce the risk for immunological interactions with patients. Conduit(s) according to the invention may be prepared by medical staff well in advance of their utilization, either by the use of endogenous blood products or by the use of commercially available tissue glue products. Naturally, exogenous blood plasma products may be used. Conduits may also be prepared on an industrial scale, for example from exogenous blood plasma products.

In one embodiment, the nerve repair conduit is in the form of a sheet or a tube. A sheet of the nerve repair conduit may be formed into any suitable and desirable shape. In another embodiment, the nerve repair conduit additionally comprises a serine protease, Factor XIII, and/or calcium ions. Calcium ions induce the formation of fibrin, whereas Factor XIII is responsible for cross-linking fibrin for the formation of a clot.

Conversion of fibrinogen into fibrin may be accomplished by use of various enzymatic cleavage systems. Consequently, in one embodiment, the serine protease is chosen from the group consisting of thrombin, plasmin, elastases, and plasminogen activators, or combinations thereof. Said serine proteases are known for modulating the conversion of fibrinogen into fibrin, or directly convert fibrinogen into fibrin.

In one embodiment, the serine protease is thrombin.

In yet another embodiment, the nerve repair conduit is loaded with Schwann cells, and/or stem cells and/or growth factors. These cell types and growth factors may help improve nerve regeneration. The Schwann cells may be autologous or heterologous.

According to a second aspect of the current invention, a method of producing a nerve repair conduit is provided, comprising the steps of

(a) dispensing a fibrinogen and thrombin containing fluid into a mould equipped with a central shaft;
(b) allowing the fluid to cure;
(c) removing the cured conduit from the mould; and
(d) removing the central shaft from within the conduit to create a channel in the nerve repair conduit.

According to one embodiment of the above aspect of the invention, the serine protease that is chosen from the group consisting of thrombin, plasmin, elastases, and plasminogen activators, or combinations thereof.

According to another embodiment, the serine protease is thrombin.

According to yet an embodiment of the invention, the above aspect comprises the additional step of loading the nerve repair conduit with Schwann cells, and/or Stem cells and/or growth factors.

The conduits may be produced in any suitable manner for producing a fibrin-containing tube or sheet of desired size and shape. Examples of shapes include such with conical or cylindrical outer shape.

A third aspect of the current invention relates to use of a glue-forming fibrinogen and serine protease containing fluids for preparing a fibrin-based nerve repair conduit.

In a first embodiment of the third aspect of the invention, use is made of a serine protease that is chosen from the group consisting of thrombin, plasmin, elastases, and plasminogen activators, or combinations thereof.

In a second embodiment of the third aspect of the invention, the serine protease is thrombin.

A fourth aspect of the current invention pertains to a method of treating nerve damage by placing a fibrin-based nerve repair conduit according to the current invention around at least one nerve stump and allowing the nerve repair conduit to guide the nerve stump during regeneration. In most cases the nerve repair conduit will be used to bridge a proximal and a distal nerve stump.

An embodiment of this last aspect of the invention comprises the additional step of loading the nerve repair conduit with Schwann cells and/or stem cells and/or growth factors. This is to further improve the nerve regeneration.

The invention thus pertains to a fibrin-based tissue engineering material for a nerve repair conduit to bridge a nerve gap. The beneficial effect of a fibrin-based nerve repair conduit on short term axon regeneration has been proved in vivo. The nerve repair conduit enabled good cell integration in vivo, and moreover allowed Schwann cells or Stem cells to retain their morphology and adhere, differentiate and proliferate on such a surface of fibrin in vitro.

Nerve repair conduits according to the invention may be fabricated from fibrinogen and thrombin containing fluids, e.g. from conventional, commercially available fibrin-based tissue glue, below referred to as fibrin glue (e.g. Tisseel®) according to the Examples below. Fibrin glue is used widely in surgical practice¹⁹. It helps surgeons to adapt tissues and to obtain haemostasis in difficult situations. It has also been used in coaptation of nerve-ends with good results^{20, 21}. Fibrin glue can be fabricated autologously from individual donors and is alternatively commercially available from different companies for clinical use²². Fibrin glue can be diluted specifically to change its dissolving and coagulation characteristics²³. Fibrin glue also has good biocompatibility and has been used also in bone tissue engineering²⁴. The important active components in conventional fibrin glue, i.e. fibrinogen and thrombin, are also commercially available separately.

A stable nerve repair conduit that guided the nerve for the initial phase of nerve regeneration was thus constructed. The nerve repair conduit supported axonal sprouting, without the central channel therein collapsing. The open channel does not impede sprouting axons and allows the nerve repair conduit to provide a permissive environment for regeneration. Due to the adhesive capability of the fibrin-based nerve repair conduit, fixation by way of sutures is not mandatory, but may be employed to equalise conditions at the ends of the nerve repair conduits. Histological analysis of the fibrin-based nerve repair conduit revealed its porous structure which would allow neurotropic growth factors to pass into the central channel of the nerve repair conduit. Conversely, the surface of the fibrin clot is cell repellent and prevents fibrous tissue to invade the channel³¹. Therefore the sprouting axons are provided with stimulating factors but not blocked by cell invasion.

Recently, it has been shown using an experimental rabbit model that autologous fibrin glue is beneficial for peripheral nerve regeneration³². Schwann cells suspended in 1% fibrinogen, 2% CaCl, 2% gentamycin and 2% of aprotinin can be added to the spinal cord of a rat and are able to proliferate³³. In human cell studies fibrin has been used successfully as a matrix for Scwann and Stem cells where an optimal concentration of fibrinogen and thrombin has been evaluated that allows a good proliferation and fast resorption of the matrix²³. These and other findings presented in this patent application suggest an additional significant advantage of the new fibrin-based nerve repair conduit for the initial phase of peripheral nerve regeneration.

The combined properties of fibrin-based nerve repair conduit according to the current invention are not matched by any other previously described autologous material including collagen, carbonate, or alginate³⁴⁻³⁶. The invention thus provides use of a fibrin-based nerve repair conduit as an improved graft to bridge peripheral nerve lesions. This fibrin-based nerve repair conduit is based on a. fibrin-based tissue glue (fibrin glue).

The present invention will now be described in the following examples with reference to the accompanying figures. The examples shall merely be seen as an illustration of the spirit and scope of the current invention, and in no way whatsoever as a limitation.

### SHORT DESCRIPTION OF THE DRAWINGS

FIG. 1 is a picture that shows fabrication of fibrin-containing nerve repair conduits using a two compound tissue glue and eight moulds equipped with central shafts in a specially fabricated compactor with a silicone inlay for fabrication of tubular conduits.

FIG. 2 is a picture that shows a produced nerve repair conduit with a central shaft.

FIG. 3 is a picture that shows a nerve repair conduit and a removed shaft

FIG. 4 is a picture that shows four moulds equipped with central shafts in a specially fabricated compactor with a silicone inlay for fabrication of chuck cone-shaped nerve repair conduits.

FIG. 5 is a diagram showing quantification of regeneration distances measured with PGP9.5 and S100 staining. The diagram shows significant improvement in axon regeneration distance as well as for the Schwann cell invasion for the fibrin-containing conduit group.

### EXAMPLES

### Example 1

### Prototype Example: Preparation of Nerve Repair Conduits

For comparative purposes, PHB nerve repair conduits were prepared from PHB sheets (Astra Tech, Sweden) that were wrapped around an intravenous cannula (16G Abbocath®, Abbott, Ireland) and heat-sealed to form nerve repair conduits for bridging a nerve defect.²⁶

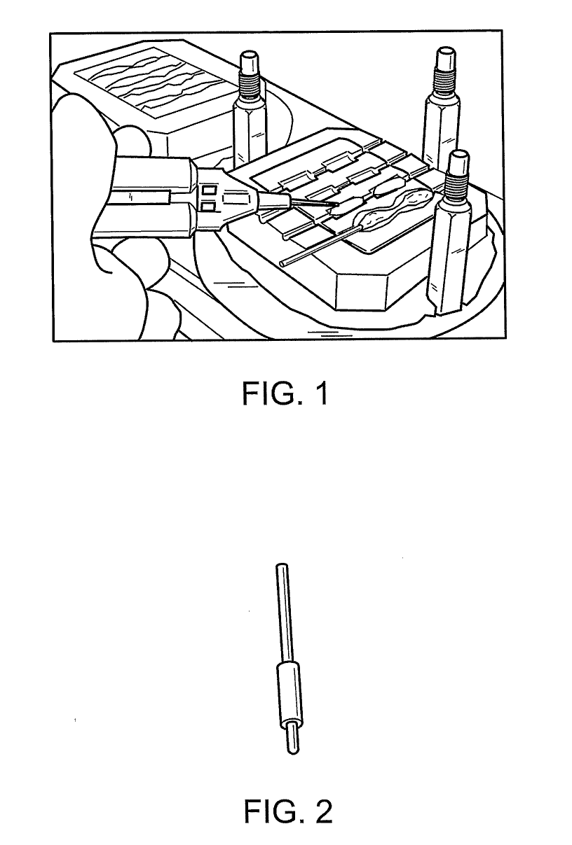

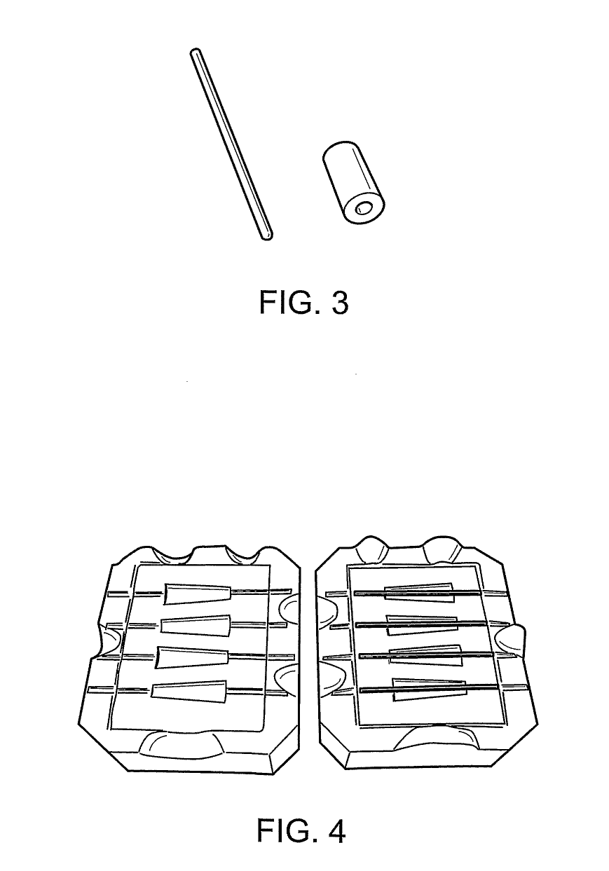

Fibrin-based nerve repair conduits according to the invention were prepared from two compound fibrin glue (Tisseel® Kit VH 1.0, Baxter SA, Switzerland). Tisseel® contains fibrinogen, 70-110 mg/mL; plasma fibronectin, 2-9 mg/mL; factor XIII, 10-50 U/mL; plasminogen, 40-120 μg/mL, aprotinin solution 3 000 KIU/ml, thrombin 4 IU/mL, calcium chlorides 40 mmol/L. Fibrin glue was filled into a specially designed mould consisting of a silicone inlay (Dublisil 20®, Dreve-Dentamid GMBH, Unna, DE) surrounded by a cast (Jade-Stone®, Whip Mix Corp. Louisville, USA) of a developed medical compressor (FIG. 1). A shaft (stainless steel, 50 mm×2 mm) was centrally embedded in the silicone inlay, receiving support at its two opposite ends and still allowing the fibrin glue access to the central cavity delimited by the inlay and surrounding the shaft. After the central cavity was filled with fibrin glue, the set of two opposite inlays with surrounding casts were joined together by use of two stainless steel cores fitted with interlocking screws, whereby nerve repair conduits were pressed into shape (FIG. 1). After 30 seconds of compression using a 5 Newton compression screw, the nerve repair conduits can be harvested. After curing, nerve repair conduits were easily removed from the silicone inlay by manipulation of the central shafts that extended beyond the cavity (FIG. 2). In this way, it was ensured that the nerve repair conduits remained intact and were not damaged. The central shaft is easily removed to provide the finished conduit (FIG. 3). The silicone inlay used in this example was provided with four parallel cavities; however the number of cavities may naturally be changed according to preferences.

The nerve repair conduits can be stored in a storage device, consisting of a simple plate with apertures for the shafts, whereby the shafts surrounded by the nerve repair conduits are stored longitudinally. In association with the storage, it is possible to put the nerve repair conduits in contact with for example a growth factor solution to let them soak with growth factors. The nerve repair conduits can be easily slipped from the stainless steel shafts (50 mm×2 mm) and thus provide a stable nerve repair conduit for implantation.

The method described can be used for manufacture of nerve repair conduits with a conical (chuck cone) (FIG. 4) or circular cylindrical (FIG. 3) outer diameter, and a uniform, circular inner diameter. Thus, the method allowed the manufacture of uniform nerve repair conduits for animal experiments with a length of 14 mm, a 1 mm wall thickness and a lumen of 2 mm. The compressor device may be designed for any length required, e.g. for longer gaps up to for example 4 cm. Also the lumen may be adjusted according to preferences.

### Example 2

### Implantation of Nerve Repair Conduits

All animal experiments were performed according to the Animal Scientific Procedures Act of 1986. The PHB and fibrin-based nerve repair conduits were prepared under aseptic conditions and designed to bridge a 10 mm gap in the left sciatic nerve of adult Sprague Dawley® rats (Harlan, UK). Surgical implantation of the nerve repair conduits was carried out by fixation of the nerve ends in the conduit with a single epineural nylon suture (9/0 Prolen, Ethicon) using an operating microscope (Zeiss, Germany). Both types of nerve repair conduit (PHB and fibrin containing nerve repair conduits, respectively) were easy to handle and the fixation with an epineural suture was uneventful. All animals used survived and displayed no autotomy. The two groups of nerve repair conduits (n=6 for each group) were implanted for two weeks whereupon they were harvested together with the proximal and distal nerve stumps. The fibrin-containing conduit (manufactured from Tisseel®) showed good integration and partial degradation in rat after two weeks.

### Example 3

### Immunohistochemistry

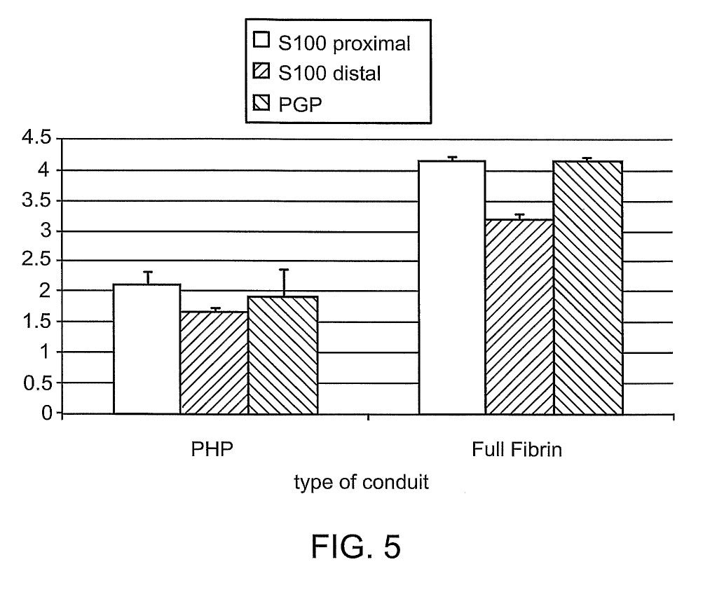

Harvested nerve repair conduits were fixed with paraformaldehyde for 16 h and then embedded in OCT freezing media and stored at −40° C. until use. Longitudinal cryo-sections (15 μm) were prepared onto Vectabond® (Vector Labs) coated slides. Sections were blocked in normal goat serum for 1 h and then incubated overnight with primary antibodies S100 (1:200, Dako) and PGP 9.5 (1:1500, Dako). The following day after 3×10 min washes in PBS, sections were incubated with secondary antibody CY3-conjugated goat anti-rabbit (1:200, Amersham Biosciences, UK) at room temperature for 2 h. The slides were mounted with Vectashield with DAPI (4′,6-diamidino-2-phenylindole) (Vector Labs, UK). The distances of axonal regeneration (PGP9.5) and SC invasion inside the transplanted nerve repair conduit (S100) were measured in mm using microscope with 4× magnification. The results are presented in FIG. 5.

### Example 4

### Statistical Analysis

Kruskal Wallis one-way ANOVA with Dunns multiple comparisons were used to statistically analyze data (SigmaStat 3.1®, SysStat Inc., London, UK). Significance was determined as *P<0.05.

### Example 5

### Morphology

Comparisons were made between PHB and fibrin-based nerve repair conduits. Both nerve repair conduits were embedded and showed no signs of haematoma or infection. Histological examination of fibrin-based nerve repair conduits after one week in vitro revealed an intact structure with obvious porosity. After 2 weeks in vivo, fibrin-based nerve repair conduits were partially resorbed, with an approximate 20% reduction in the diameter of nerve repair conduit. PHB nerve repair conduits showed no signs of hydrolytic degradation.

### Example 6

### Axon Regeneration and Schwann Cell Invasion

After 2 weeks, nerves remained attached to the nerve repair conduits and showed a growth cone directed towards the distal end. PGP9.5 immunohistochemistry indicated that the fibrin-based nerve repair conduit showed significantly better axon regeneration distance than the PHB nerve repair conduit (1.9 mm vs. 4.1 mm respectively, P<0.05) (FIG. 5). S100 staining showed that axons which had not passed through the centre of the nerve repair conduit did not cause the fibrin-based nerve repair conduit to collapse. A distinct area was identified where the nerve repair conduit retains its lumen after the two weeks in vivo. This indicates that the fibrin-based nerve repair conduit can provide sufficient rigidity in vivo to guide the regenerating nerve. The Schwann cell regeneration was assessed proximally and distally using S100 staining, showing a superior cell intrusion within the fibrin-based nerve repair conduit proximally (S100; 2.1 mm vs. 4.2 mm) and distally (S100; 1.7 mm vs. 3.2 mm).

### LITERATURE

1. Millesi H. Factors affecting the outcome of peripheral nerve surgery. Microsurgery 2006: 26: 295-302.
2. Young V L, Wray R C, Weeks P M. The results of nerve grafting in the wrist and hand. Ann Plast Surg 1980: 5: 212-5.
3. Dellon A L, Jabaley M E. Reeducation of sensation in the hand following nerve suture. Clin Orthop Relat Res 1982: 75-9.
4. Beazley W C, Milek M A, Reiss B H. Results of nerve grafting in severe soft tissue injuries. Clin Orthop Relat Res 1984: 208-12.
5. Mackinnon S E, Dellon A L. A comparison of nerve regeneration across a sural nerve graft and a vascularized pseudosheath. J Hand Surg [Am] 1988: 13: 935-42.
6. Keskin M, Akbas H, Uysal O A, et al. Enhancement of nerve regeneration and orientation across a gap with a nerve graft within a vein conduit graft: a functional, stereological, and electrophysiological study. Plast Reconstr Surg 2004: 113: 1372-9.
7. Battiston B, Geuna S, Ferrero M, Tos P. Nerve repair by means of tubulization: literature review and personal clinical experience comparing biological and synthetic conduits for sensory nerve repair. Microsurgery 2005: 25: 258-67.
8. Evans P J, Midha R, Mackinnon S E. The peripheral nerve allograft: a comprehensive review of regeneration and neuroimmunology. Prog Neurobiol 1994: 43: 187-233.
9. Lundborg G, Dahlin L B, Danielsen N. Ulnar nerve repair by the silicone chamber technique. Case report. Scand J Plast Reconstr Surg Hand Surg 1991: 25: 79-82.
10. Zhao Q, Lundborg G, Danielsen N, Bjursten L M, Dahlin L B. Nerve regeneration in a ‘pseudo-nerve’ graft created in a silicone tube. Brain Res 1997: 769: 125-34.
11. He F C, Fan Q Y, Cushway T R, et al. Long-term result of guided nerve regeneration with an inert microporous polytetrafluoroethylene conduit. Chin J Traumatol 2003: 6: 145-51.
12. Lanzetta M, Gal A, Wright B, Owen E. Effect of FK506 and basic fibroblast growth factor on nerve regeneration using a polytetrafluoroethylene chamber for nerve repair. Int Surg 2003: 88: 47-51.
13. Lundborg G. Neurotropism, frozen muscle grafts and other conduits. J Hand Surg [Br] 1991: 16: 473-6.
14. Merle M, Dellon A L, Campbell J N, Chang P S. Complications from silicon-polymer intubulation of nerves. Microsurgery 1989: 10: 130-3.
15. Gibson K L, Remson L, Smith A, et al. Comparison of nerve regeneration through different types of neural prostheses. Microsurgery 1991: 12: 80-5.
16. Crawley W A, Dellon A L. Inferior alveolar nerve reconstruction with a polyglycolic acid bioabsorbable nerve conduit. Plast Reconstr Surg 1992: 90: 300-2.
17. Mackinnon S E, Dellon A L. Clinical nerve reconstruction with a bioabsorbable polyglycolic acid tube. Plast Reconstr Surg 1990: 85: 419-24.
18. Mohanna P N, Terenghi G, Wiberg M. Composite PHB-GGF conduit for long nerve gap repair: a long-term evaluation. Scand J Plast Reconstr Surg Hand Surg 2005: 39: 129-37.
19. Albala D M, Lawson J H. Recent clinical and investigational applications of fibrin sealant in selected surgical specialties. J Am Coll Surg 2006: 202: 685-97.
20. Ornelas L, Padilla L, Di Silvio M, et al. Fibrin glue: an alternative technique for nerve coaptation—Part I. Wave amplitude, conduction velocity, and plantar-length factors. J Reconstr Microsurg 2006: 22: 119-22.
21. Ornelas L, Padilla L, Di Silvio M, et al. Fibrin glue: an alternative technique for nerve coaptation—Part II. Nerve regeneration and histomorphometric assessment. J Reconstr Microsurg 2006: 22: 123-8.
22. Le Guehennec L, Layrolle P, Daculsi G. A review of bioceramics and fibrin sealant. Eur Cell Mater 2004: 8: 1-10; discussion 10-1.
23. Bensaid W, Triffitt J T, Blanchat C, et al. A biodegradable fibrin scaffold for mesenchymal stem cell transplantation. Biomaterials 2003: 24: 2497-502.
24. Fang H, Peng S, Chen A, et al. Biocompatibility studies on fibrin glue cultured with bone marrow mesenchymal stem cells in vitro. J Huazhong Univ Sci Technolog Med Sci 2004: 24: 272-4.
25. Tohill M P, Mann D J, Mantovani C M, Wiberg M, Terenghi G. Green fluorescent protein is a stable morphological marker for schwann cell transplants in bioengineered nerve conduits. Tissue Eng 2004: 10: 1359-67.
26. Hazari A, Wiberg M, Johansson-Ruden G, Green C, Terenghi G. A resorbable nerve conduit as an alternative to nerve autograft in nerve gap repair. Br J Plast Surg 1999: 52: 653-7.
27. Belkas J S, Munro C A, Shoichet M S, Johnston M, Midha R. Long-term in vivo biomechanical properties and biocompatibility of poly(2-hydroxyethyl methacrylate-co-methyl methacrylate) nerve conduits. Biomaterials 2005: 26: 1741-9.
28. Belkas J S, Munro C A, Shoichet M S, Midha R. Peripheral nerve regeneration through a synthetic hydrogel nerve tube. Restor Neurol Neurosci 2005: 23: 19-29.
29. Belkas J S, Shoichet M S, Midha R. Peripheral nerve regeneration through guidance tubes. Neurol Res 2004: 26: 151-60.
31. Takazawa R, Yamato M, Kageyama Y, Okano T, Kihara K. Mesothelial cell sheets cultured on fibrin gel prevent adhesion formation in an intestinal hernia model. Tissue Eng 2005: 11: 618-25.
32. Choi B H, Han S G, Kim S H, et al. Autologous fibrin glue in peripheral nerve regeneration in vivo. Microsurgery 2005: 25: 495-9.
33. Blits B, Carlstedt T P, Ruitenberg M J, et al. Rescue and sprouting of motoneurons following ventral root avulsion and reimplantation combined with intraspinal adeno-associated viral vector-mediated expression of glial cell line-derived neurotrophic factor or brain-derived neurotrophic factor. Exp Neurol 2004: 189: 303-16.
34. Kim D H, Connolly S E, Zhao S, et al. Comparison of macropore, semipermeable, and nonpermeable collagen conduits in nerve repair. J Reconstr Microsurg 1993: 9: 415-20.
35. Mackinnon S E, Dellon A L. A study of nerve regeneration across synthetic (Maxon) and biologic (collagen) nerve conduits for nerve gaps up to 5 cm in the primate. J Reconstr Microsurg 1990: 6: 117-21.
36. Suzuki Y, Tanihara M, Ohnishi K, et al. Cat peripheral nerve regeneration across 50 mm gap repaired with a novel nerve guide composed of freeze-dried alginate gel. Neurosci Lett 1999: 259: 75-8.

1. A bioresorbable fibrin-based nerve repair conduit in the form of a sheet produced from tissue glue.

2. (canceled)

3. The nerve repair conduit according to claim 1, wherein the nerve repair conduit additionally comprises a serine protease, and/or Factor XIII, and/or calcium ions.

4. The nerve repair conduit according to claim 3, wherein the serine protease is chosen from the group consisting of trombin, plasmin, elastases, and plasminogen activators, and combinations thereof.

5. The nerve repair conduit according to claim 3, wherein the serine protease is thrombin.

6. The nerve repair conduit according to claim 1, wherein the nerve repair conduit is loaded with Schwann cells, and/or Stem cells and/or growth factors.

7. A method of producing a fibrin-based nerve repair conduit comprising the steps of
(a) dispensing tissue glue forming fibrinogen and serine protease containing fluids into a mould equipped with a central shaft;
(b) allowing the fluid to cure;
(c) removing the cured conduit from the mould; and
(d) removing the central shaft from within the conduit to create a channel in the nerve repair conduit.

8. The method of producing a nerve repair conduit according to claim 7, wherein the serine protease is chosen from the group consisting of thrombin, plasmin, elastases, and plasminogen activators, and combinations thereof.

9. The method of producing a nerve repair conduit according to claim 8, wherein the serine protease is thrombin.

10. The method according to claim 7, comprising the additional step of loading the nerve repair conduit with Schwann cells, and/or Stem cells and/or growth factors.

11. Use of glue-forming fibrinogen and serine protease containing fluids for preparing a fibrin-based nerve repair conduit.

12-13. (canceled)

14. A method of treating nerve damage by placing a fibrin-based nerve repair conduit claimed in claim 1 around at least one nerve stump and allowing the nerve repair conduit to guide the nerve stump during regeneration.

14. The nerve repair conduit according to claim 3, wherein the nerve repair conduit is loaded with Schwann cells, and/or Stem cells and/or growth factors.

15. The nerve repair conduit according to claim 4, wherein the nerve repair conduit is loaded with Schwann cells, and/or Stem cells and/or growth factors.

16. The nerve repair conduit according to claim 5, wherein the nerve repair conduit is loaded with Schwann cells, and/or Stem cells and/or growth factors.

17. The method according to claim 8, comprising the additional step of loading the nerve repair conduit with Schwann cells, and/or Stem cells and/or growth factors.

18. The method according to claim 9, comprising the additional step of loading the nerve repair conduit with Schwann cells, and/or Stem cells and/or growth factors.

19. A method of treating nerve damage by placing a fibrin-based nerve repair conduit claimed in claim 2 around at least one nerve stump and allowing the nerve repair conduit to guide the nerve stump during regeneration.

20. A method of treating nerve damage by placing a fibrin-based nerve repair conduit claimed in claim 3 around at least one nerve stump and allowing the nerve repair conduit to guide the nerve stump during regeneration.

21. A method of treating nerve damage by placing a fibrin-based nerve repair conduit claimed in claim 4 around at least one nerve stump and allowing the nerve repair conduit to guide the nerve stump during regeneration.

22. A method of treating nerve damage by placing a fibrin-based nerve repair conduit claimed in claim 5 around at least one nerve stump and allowing the nerve repair conduit to guide the nerve stump during regeneration.
